(19) United States
(12) Patent Application Publication
(10) Pub. No.: US 2007/0045039 A1
(43) Pub. Date: Mar. 1, 2007

(54) Cover for the diaphragm of a stethoscope and dispenser for such a cover

(76) Inventors: Arjang Agahi, Dunadry County Antrim (GB); Orang Agahi, Belfast (GB)

Correspondence Address: VAN DYKE, GARDNER, LINN AND BURKHART, LLP, 2851 CHARLEVOIX DRIVE, S.E., P.O. BOX 888695, GRAND RAPIDS, MI, 49588-8695, US

(21) Appl. No.: 10/573,441

(22) PCT Filed: Sep. 24, 2004
(86) PCT No.: PCT/EP04/10838

§ 371 Date: Mar. 24, 2006

(30) Foreign Application Priority Data
Sep. 25, 2003 (GB) 0322478.9

(51) Int. Cl. A61B 7/02

(52) U.S. Cl. 181/131

(57) ABSTRACT

An apparatus for dispensing a cover device, the cover device comprising a frame and a membrane. The cover device is actuatable from a first state, in which an outer portion of the membrane is seated on, or around, an outer surface of the frame, and a second state in which the outer portion of the membrane is located generally inwardly of its seated position. The apparatus comprises a magazine for storing at least one cover device in said first state, the magazine having a mouth through which the at least one cover device may be dispensed; and means for actuating the at least one stored cover device from the first state to the second state. In a preferred embodiment, said actuating means actuates the at least one stored cover device from the first state to the second state upon movement of said at least one stored cover device into the magazine. Hence, when the head of a stethoscope (or other instrument) is inserted into the mouth of the dispenser, thereby pushing the topmost cover device into the magazine in a direction generally away from the mouth, the cover device is actuated to the second state thereby causing it to grip the head of the stethoscope (or other instrument).

### FIELD OF THE INVENTION

One aspect of the present invention relates to a cover device for an instrument particularly, but not exclusively, a medical instrument such as a stethoscope. Another aspect of the invention relates to an apparatus for dispensing cover devices, especially medical instrument cover devices.

### BACKGROUND TO THE INVENTION

A person's body harbours various types of bacteria and other micro-organisms many of which are infectious. The use of a medical instrument, and in particular a stethoscope, on more than one patient can cause cross-contamination of patients. By way of example, this is a particular concern in cases where the instrument comes into contact with blood.

It is good practice to clean the instrument between patients but often cleaning is not thoroughly performed, or is not performed at all.

To address this problem, it is known to provide a protective cover for stethoscopes. For example, U.S. Pat. No. 5,365,023 (Lawton) discloses a stethoscope cover comprising an elastic membrane with a rolled rim. In use, the rim is stretched over the head of the stethoscope and the membrane is held in place by its elastic resilience. U.S. Pat. No. 5,813,992 (Henwood) discloses a bag-type stethoscope cover which, in use, encases the entire stethoscope head.

Both of these known covers are considered to be cumbersome to fit onto and to remove from the stethoscope head.

It would be desirable therefore to provide a cover for protecting the head of a medical instrument, particularly a stethoscope, which is relatively simple to fit and remove. It would also be desirable to provide an apparatus for dispensing same.

### SUMMARY OF THE INVENTION

Accordingly, a first aspect of the invention provides an apparatus for dispensing a cover device, the cover device comprising a frame and a membrane, the cover device being actuatable from a first state, in which an outer portion of the membrane is seated on, or around, an outer surface of the frame such that the frame holds the membrane in a generally deployed state, and a second state in which the outer portion of the membrane is located generally inwardly of its seated position, the apparatus comprising: a magazine for storing at least one cover device in said first state, the magazine having a mouth through which the at least one cover device may be dispensed; and means for actuating the at least one stored cover device from the first state to the second state. The outer portion may be integrally formed with the membrane, in which case it may be said to comprise part of the membrane, or it may be fixed to the membrane. In either case it may be said that the outer portion is associated with the membrane.

In a preferred embodiment, said actuating means actuates the at least one stored cover device from the first state to the second state upon movement of said at least one stored cover device into the magazine. Hence, when the head of a stethoscope (or other instrument) is inserted into the mouth of the dispenser, thereby pushing the topmost cover device into the magazine in a direction generally away from the mouth, the cover device is actuated to the second state thereby causing it to grip the head of the stethoscope (or other instrument).

Advantageously, an actuating mechanism is provided in the apparatus and is arranged to urge the at least one cover device towards the mouth. Conveniently, the actuating mechanism comprises a platform mounted on a spring, the spring being biased to urge the platform towards the mouth.

Preferably, a retaining lip is provided around at least part of the mouth, the retaining lip extending inwardly of the mouth. The retaining lip is typically dimensioned to engage with, during use, the outer portion of the cover device nearest the mouth when in said first state, thereby retaining said cover device in the apparatus, and to allow said cover device to be removed from the apparatus when in the second state.

In a preferred embodiment, the actuating means comprises means for dislodging the outer portion of the membrane from its seat on the frame. The dislodging means is advantageously actuatable between a dislodging state and a deflected state. Preferably, the dislodging means is actuatable from said dislodging state to said deflected state by passage of a cover device moving towards said mouth. The dislodging means is preferably biased to adopt the dislodging state.

In a preferred embodiment, the dislodging means comprises at least one projection extending away from an interior surface of the magazine and being spaced-apart from the mouth. The at least one projection may extend obliquely from the interior surface of the magazine in a direction generally towards the mouth. Advantageously, at least a part of said projections are formed from a flexible, resilient material. The at least one projection may be carried by a ring, the ring being seated on a shoulder formed at the interior surface of the magazine.

In an alternative embodiment, the apparatus comprises a collar, the magazine being movable with respect to the collar in a first direction, and wherein the collar carries said actuating means, said actuating means actuating the at least one stored cover device from the first state to the second state upon movement of said magazine in said first direction. Preferably, resilient biasing means are provided between said collar and said magazine, the resilient biasing means being arranged to urge said magazine in a second direction, said second direction being generally opposite to said first direction. Preferably, said collar is located at least partially around the external periphery of the magazine.

A second aspect of the invention provides a cover device comprising a frame and a membrane, the cover device being actuatable from a first state, in which an outer portion of the membrane is seated on an outer surface of the frame such that the frame holds the membrane in a generally deployed state, and a second state in which the outer portion of the membrane is located generally inwardly of its seated position.

Preferably, the outer portion of the membrane comprises a peripheral ring. The peripheral ring, or other outer portion, may be integrally formed with the membrane, in which case it may be said to comprise part of the membrane, or it may be fixed to the membrane. In either case it may be said that the outer portion is associated with the membrane.

Preferably, the outer surface of the frame is concave in transverse cross-sectional profile to define a seat for the outer portion of the membrane.

Advantageously, at least the outer portion of the membrane is formed from elastic material.

In a preferred embodiment, in the first state, said outer portion is stretched by the frame such that the membrane is self-retaining on the frame. More preferably, in the second state, said outer portion is contracted with respect to the first state (under its inherent resilience since it is dislodged from its seat on the frame) and is located generally inwardly of the frame. This enables the membrane, and more particularly its outer portion, to grip the head of a stethoscope (or other instrument) and be self-retaining thereon.

A third aspect of the invention provides a dispensing system comprising said dispensing apparatus and one or more cover devices.

Further advantageous aspects of the invention will become apparent to those ordinarily skilled in the art upon review of the following description of a specific embodiment and with reference to the accompanying drawings.

### BRIEF DESCRIPTION OF THE DRAWINGS

Embodiments of each aspect of the invention are now described by way of example and with reference to the accompanying drawings in which like numerals are used to indicate like parts and in which:

FIG. 1 presents a perspective view of a cover device embodying one aspect of the invention fitted onto a stethoscope head, and a dispensing apparatus embodying a further aspect of the invention;

FIG. 2 presents a cut-away perspective view of the dispensing apparatus shown in FIG. 1;

FIG. 3 presents a cut-away side view of the dispensing apparatus and an uncovered stethoscope head;

FIG. 4 presents a cut-away perspective view of part of the dispensing apparatus of FIG. 3;

FIG. 5 presents a cut-away side view of the dispensing apparatus and a stethoscope head in the mouth of the apparatus;

FIG. 6 presents a cut-away perspective view of part of the dispensing apparatus of FIG. 5;

FIG. 7 presents a cut-away side view of the dispensing apparatus and a stethoscope head fitted with a cover device in the mouth of the apparatus;

FIG. 8 presents a cut-away perspective view of part of the dispensing apparatus of FIG. 7;

FIG. 9 presents a cut-away perspective view of an alternative embodiment of a dispensing apparatus containing a plurality of cover devices;

FIG. 10 presents a cut-away perspective view of a portion of the dispensing apparatus of FIG. 9;

FIG. 11 is a cut-away side view of a portion of the dispensing apparatus of FIG. 9 showing part of a stethoscope head inserted in the topmost cover device; and

FIG. 12 is a cut-away side view of the portion shown in FIG. 11 showing the topmost cover device fitted to the stethoscope head.

### DETAILED DESCRIPTION OF THE DRAWINGS

The following description is made in the context of stethoscopes although it will be understood that the invention is not limited to use with stethoscopes and may alternatively be adapted for use with other medical or non-medical instruments.

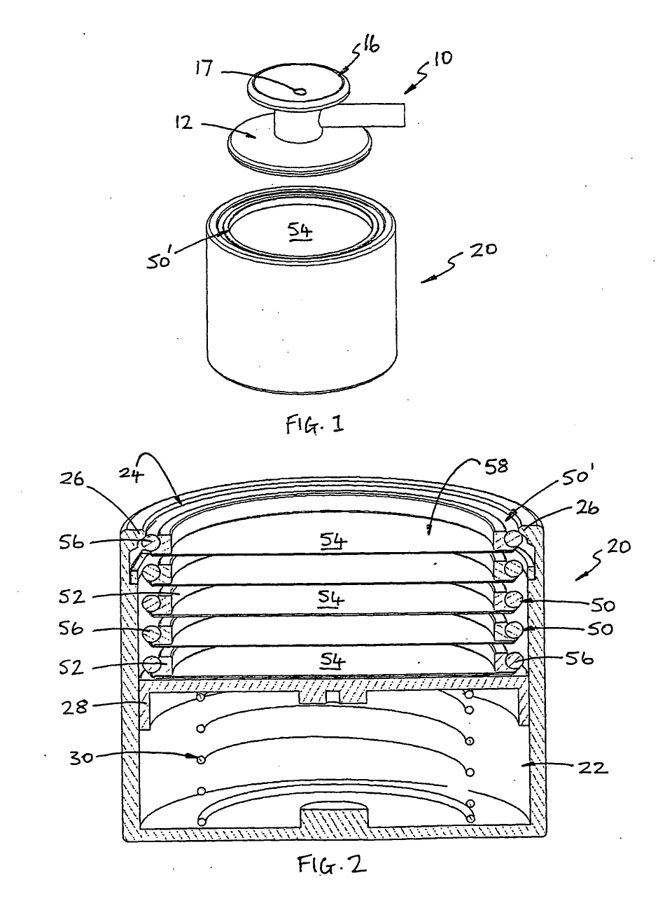

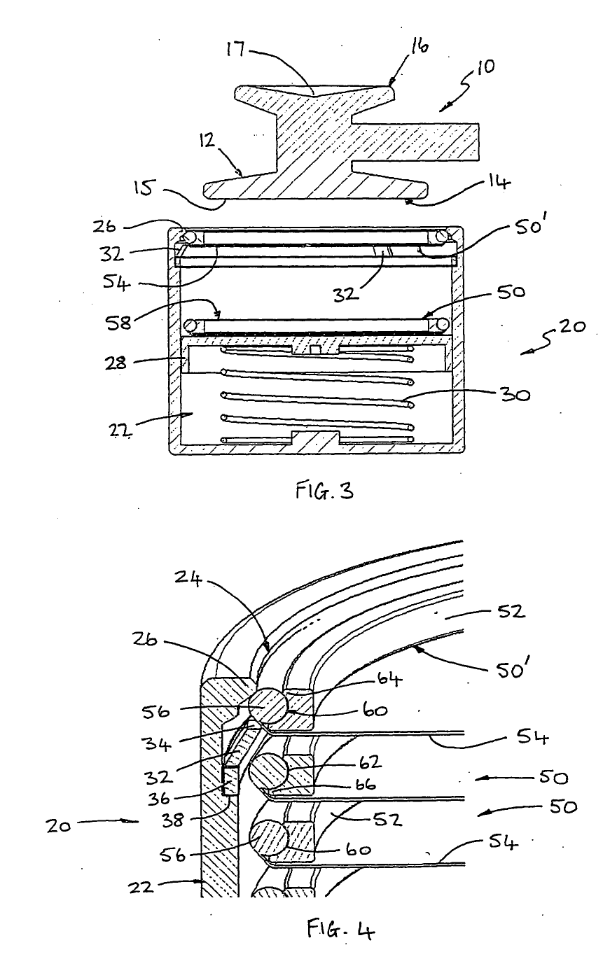

Stethoscopes are well known and normally include a head comprising one or more acoustic transponders. FIGS. 1 and 3 best illustrate a typical stethoscope head 10 comprising a first acoustic transponder in the form of a cup or cone portion 12 the mouth of which is covered by a diaphragm 14. Such acoustic transponders are commonly referred to as diaphragm transponders. The head 10 also comprises a second acoustic transponder in the form of an open-mouthed bell 16. Such acoustic transponders are commonly referred to as bell transponders. The diaphragm transponder 12 is more commonly used than the bell transponder 16 and it is therefore considered to be of primary importance to provide a cover for the diaphragm transponder 12. Moreover, since, during use, it is the exterior surface 15 of the diaphragm 14 which comes into contact with a patient, it is considered to be particularly important to cover the exterior surface 15 of the diaphragm 14.

Referring now to FIGS. 1 and 2, there is shown, generally indicated at 20, an apparatus for dispensing one or more cover devices 50 which are particularly suited for protecting the diaphragm transponder 12 and, more particularly, the exterior surface 15 of the diaphragm 14.

Each cover device 50 comprises a frame 52 for carrying a membrane 54. The membrane 54, or sheath, may be formed from any suitable material, especially rubber, e.g. silicone, or plastics, and is preferably flexible and resilient, or elastic. Moreover, the membrane material is advantageously selected so as to minimise acoustic attenuation while presenting a barrier to micro-organisms and other contaminants. For example, the membrane 54 may be formed from latex or silicone, or similar material, and may be approximately 0.1-0.2 mm in thickness.

The frame 52 may be formed from substantially rigid, or semi-rigid, material, such as plastics, paper, card or a paper or card based material. The frame 52 is a self-supporting structure which, when the cover device 50 is in a first state wherein the membrane is fitted to the frame 52 (as depicted in FIGS. 2 to 6), serves to hold the membrane 54 in a deployed, or at least a generally deployed, state. When the membrane 54 is formed from flexible resilient material, the relative dimensions of the frame 52 and membrane 54 are preferably such that membrane 54 is held substantially taut by the frame 52 in the deployed state and is typically stretched by the frame 52. It will be understood that the membrane 54 need not necessarily be held taut when fitted to the frame 52.

The frame 52 is shaped and dimensioned to fit the instrument to which it is intended to be fitted. In the illustrated embodiment, the frame 52 is shaped and dimensioned to fit the diaphragm transponder 12 of the stethoscope head 10, and more particularly to fit over the transponder 12 such that the exterior surface 15 of the diaphragm 14 is covered by the membrane 54. Hence, the frame 52 comprises a generally circular ring. It will be understood that the frame 52 may alternatively take other shapes and dimensions to suit the particular instrument to which it is intended to be fitted.

The membrane 54 is, in a preferred embodiment, self-retaining on the frame 52 by virtue of its inherent resilience. To help retain the membrane 54 on the frame 52, the membrane 54 is preferably provided with a peripheral ring 56 (which need not necessarily be circular) formed from elastic or resilient material (typically from the same material as the membrane 54). The peripheral ring 56 may be co-formed with the membrane 54 in any suitable manner and may, for example, be formed by rolling a peripheral portion of the membrane 54, or by injection moulding. In the illustrated embodiment, the peripheral ring 56 is generally circular in shape and has a diameter less than that of the frame 52 so that the membrane 54, and in particular the ring 56, must be stretched to be fitted on the frame 52. When fitted on the frame 52, the membrane 54 covers a mouth defined by one face of the frame 52 leaving an open mouth 58 at the other face of the frame 52. It is noted that the peripheral ring 56 is viewed herein as an integral part of, and in particular as an outer portion of, the membrane 54. However, the ring 56 and membrane 54 need not necessarily be formed from the same material.

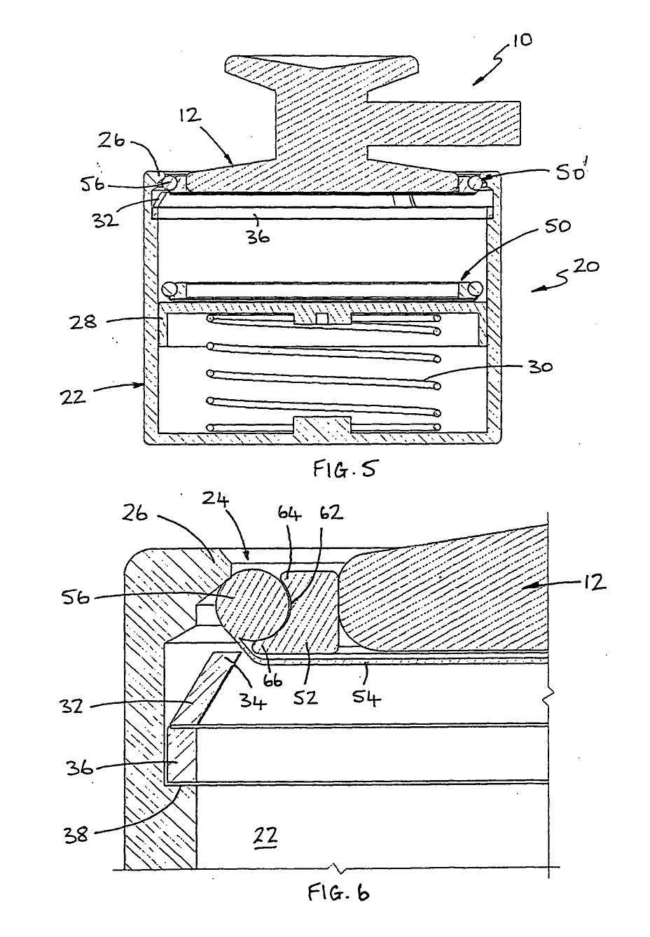

The outer periphery, or outer side, of the frame 52 is shaped to define a seat 60 for the peripheral ring 56 of the membrane 54. To this end, the outer side 62 of the frame 52 is advantageously shaped in a manner which corresponds with the profile of the peripheral ring 56. In the preferred embodiment, the peripheral ring 56 is of substantially circular cross-section and the outer side 62 of the frame 52 is correspondingly, or at least compatibly, curved. Hence, the profile, in transverse cross-section, of the outer side 62 of the frame 52 is concave. Advantageously, the outer side 62 of the frame 52 is shaped to define a first peripheral lip 64 which runs around the periphery of the frame 52 and which, conveniently, is located at or adjacent the face of the frame 52 which defines the open mouth 58. The outer side 62 is preferably shaped to define a second peripheral lip 66 which runs around the periphery of the frame 52 and which is, conveniently, located at or adjacent the opposite face of the frame 52. The second lip 66 is advantageously larger than the first lip 64, i.e. it protrudes farther from the outer side 62 than does the first lip 64. In an unfitted state (i.e. when the cover device 50 is not fitted to a stethoscope), the peripheral ring 56 is seated in the seat 60 as shown in FIGS. 2 to 6. In this state, the membrane 54 is carried by, or fitted to, the frame 52 and is held in the deployed state. In a preferred embodiment, a portion of the membrane 54 is wrapped around the peripheral ring 56 when the ring 56 is seated on the frame 52 (for example as shown in FIG. 6). The wrapped portion of the membrane 54 serves as slack and facilitates the dislodgement of the ring 56 from its seat 60 on the frame 52.

In an alternative embodiment, the frame and the membrane may be co-formed, or integrally formed, with one another as opposed to being separate as shown in FIGS. 1 to 8. Conveniently, this may be achieved by co-forming the frame and the membrane from the same material, for example rubber or plastics. In such embodiments, a reinforcing member, e.g. wire or ring (not shown), of any suitable material, e.g. metal, may be provided inside or around the frame to give strength and support to the frame so that the frame may hold the membrane in a deployed state. Alternatively still, the frame and the membrane may be bonded, or otherwise fixed, to one another.

Referring now in particular to FIG. 2, the dispensing apparatus 20, or dispenser, comprises a chamber, or magazine 22, for storing one or more cover devices 50. The magazine 22 is shaped to define a dispensing mouth 24. A lip 26 is provided at the mouth 24 and extends inwardly of the mouth 24. Preferably, the lip 26 extends around the entire, or substantially the entire, periphery of the mouth 24. Alternatively, one or more spaced-apart lip portions may be provided around the periphery of the mouth 24. As may be seen from FIG. 2, the lip 26 overhangs the interior of the magazine.

The dispenser 20 also includes dislodging means conveniently in the form of one or more projections 32. In the illustrated embodiment, the dispenser 20 comprises three projections 32 (only two visible) spaced-apart around the periphery of the dispenser 20. As may best be viewed in FIGS. 4 and 6, the projections 32 extend obliquely from the interior wall of the magazine 22 in a direction generally inwardly of the magazine and generally towards the mouth 24. The projections 32 are dimensioned so that they may interfere with, or engage with, cover devices 50 within the magazine 22. The projections 32 are located adjacent and beneath the mouth 24 of the magazine 22 so that they may interfere only with the topmost cover device 50′ (i.e. the cover device located nearest the mouth 24) as is described in more detail below.

In the drawings, the projections 32 are shown in a dislodging, or rest, state in which the respective free end 34 of each projection 32 lies beneath the topmost cover device 50′(i.e. beyond the topmost cover device 50′ with respect to the mouth 24) and overlaps with the cover device 50′ in a lateral or transverse direction. In this state, movement of the topmost cover device 50′ in a direction generally into the magazine 22 causes the peripheral ring 56 to engage with the free ends 34 of the projections 32. The projections 32 are pivotable, or otherwise movable, with respect to the magazine 22 so that they may be pushed out of the rest state towards the walls of the magazine 22. To this end, the projections 32 are conveniently formed at least partially from a flexible plastics material. The projections 32 may be co-formed with a carrier ring 36 which is seated on a shoulder 38 formed in the interior wall of the magazine 22. The carrier ring 36 and projections 32 may be formed from, for example, plastics. Alternatively, the projections 32 may be integrally formed with the magazine 22. The projections 32 are preferably resiliently biased to adopt the rest state. This may be achieved by, for example, inherent resilience of the material, e.g. plastics, used to form at least part (and conveniently the whole) of the projections 32, or alternatively by other resilient biasing means (not shown) such as a spring.

When stored in the magazine 22, the cover devices 50 are stacked one on top of the other in a substantially coaxial manner. Each cover device 50 is disposed in a respective plane which is substantially parallel with the plane of the mouth 24 of the dispenser 20. Conveniently, at least the interior walls of the magazine 22 are shaped substantially to match the shape of the frames 52 when carrying a respective membrane 54. In the illustrated embodiment, the magazine 22 is substantially cylindrical in shape.

The dispenser 20 further comprises an actuating mechanism for pushing the cover devices 50 towards the mouth 24 of the dispenser 20. The actuating mechanism comprises a platform 28 located within the magazine 22 and movable within the magazine in a direction generally towards and away from the mouth 24. The actuating mechanism also includes means for moving the platform towards the mouth 24. The moving means preferably comprises resilient biasing means, for example a spring 30, biased to urge the platform 28 towards the mouth 24. In the illustrated embodiment, the platform 23 is mounted on, or rests on, a compression spring 30. The illustrated actuating mechanism 28, 30 is therefore piston-like in form although it will be understood that it may take a variety of alternative forms.

In use, a stack of one or more cover devices 50 rests on the platform 28 and is urged towards the mouth 24 of the dispenser 20 under the action of the spring 30. The topmost cover device 50′ in the stack, i.e. the cover device 50 nearest to the mouth 24, is prevented from exiting the dispenser 20 via the mouth 24 by engagement with the lip 26. In particular (and as may best be seen from FIG. 4) the peripheral ring 56 of the membrane 54 engages with the lip 26 such that the topmost cover device 50′ is held in the mouth 24 of the dispenser 20 as shown in FIG. 2. The cover devices 50 are arranged so that their respective open mouth 58 faces the mouth 24 of the magazine 22 and is therefore exposed by, or is accessible via, the mouth 24 of the dispenser 20 when they reach the topmost position.

The operation of the dispenser 20 is now described with reference in particular to FIGS. 3 to 8. For reasons of clarity, the entire stack of cover devices 50 is not shown in FIGS. 3 to 8. FIG. 3 shows an uncovered stethoscope head 10 approaching the open mouth 24 of the dispenser 20. The topmost cover device 50′ is located in the mouth 24 of the dispenser 20 as described above. A user (not shown) pushes the stethoscope head 10 into the open mouth 24 of the dispenser 20 so that the transponder 12 fits into the open mouth 58 of the topmost cover device 50′ (see FIGS. 5 and 6).

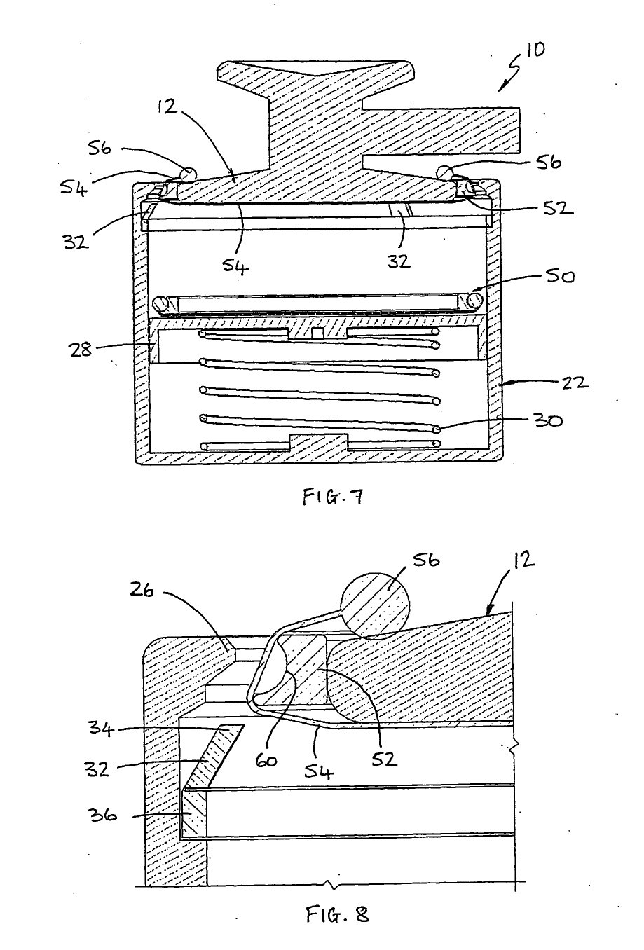

As the user continues to push the stethoscope head 10 into the dispenser 20, the diaphragm 14 engages with the membrane 24 causing the topmost cover device 50′ to be pushed into the dispenser 20 against the bias of the spring 30 (the entire stack of cover devices 50 is pushed downwardly (i.e. into the magazine 22 in a direction away from the mouth 24) as viewed in FIG. 2). As the topmost cover device 50′ moves downwardly (as viewed in FIG. 5), the peripheral ring 56 engages with the respective free ends 34 of the dislodging projections 32. The projections 32 dislodge the peripheral ring 56 from its seat 60 on the frame 52 thereby pushing it over the first lip 64, thereby causing the cover device 50′ to adopt a second state (as shown in FIGS. 1, 7 and 8). This is facilitated by the fact that the first lip 64 is relatively short. Because the peripheral ring 56 is kept under tension, i.e. stretched, when carried by the frame 52, once it is dislodged by the projections 32, it contracts under its own resilience to embrace the transponder 12, as shown in FIGS. 7 and 8. It will be seen from FIGS. 7 and 8 that, in the second state, the peripheral ring 56 is located inwardly of the frame 52 and this allows it to grip the head 10.

The cover device 50′ is thus fitted to the stethoscope head 10. The user may then withdraw the stethoscope head 10 (with fitted cover 50) from the dispenser 20. The lip 26 does not interfere with the withdrawal of the head 10 since the overall diameter of the cover device 50 in the second, or fitted, state is less than it is in the first, or unfitted, state. The fitted cover device 50 is self-retaining on the stethoscope head 10 under the resilience of the membrane 54, and in particular the peripheral ring 56. It is noted that the membrane 54 need not necessarily be formed from resilient or elastic material even when the peripheral ring is formed from resilient or elastic material.

A fitted cover device 50 may easily be removed by pulling on the peripheral ring 56. Advantageously, one or more perforations (not shown) and/or one or more tabs (not shown) may be formed in the membrane 54 and peripheral ring 56 to facilitate removal.

Once the topmost cover device 50′ has been dispensed, the next cover device 50 is pushed, under the action of the spring 30, into the topmost position. As the next cover device 50 moves into the topmost position, it engages with the projections 32. The flexibility of the projections 32 and their oblique disposition allows them to be pushed towards the walls of the magazine 22, i.e. deflected, by the passing cover device 50. Once the cover device 50 has reached the topmost position, the projections 32 adopt the rest state under their own resilience. The projections 32 may therefore be said to serve as latch means that do not prevent movement of the cover devices 50 in a direction generally towards the mouth 24 but which engages with the topmost cover device 50′ when moved away from the mouth 24.

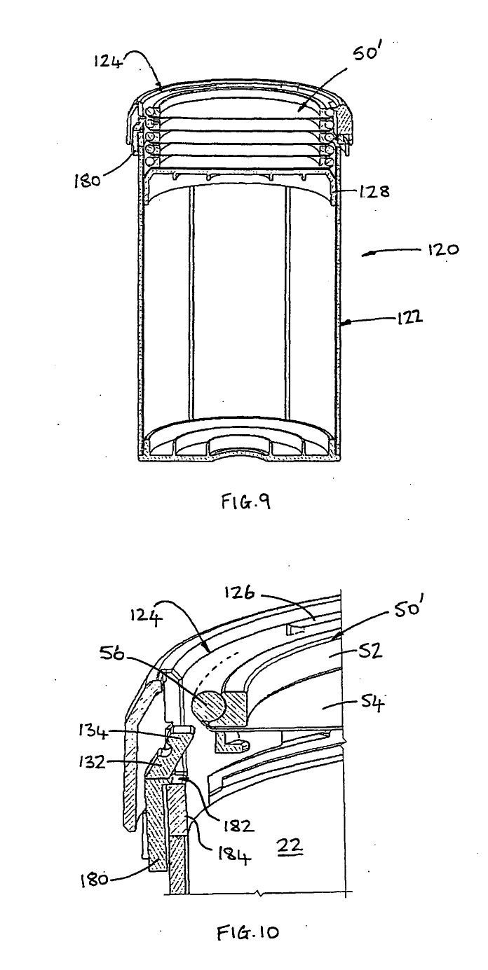

FIGS. 9 to 12 illustrate an alternative embodiment of a dispensing device, generally indicated as 120. The apparatus 120 is generally similar to the apparatus 20 and so like numerals are used to indicate like parts and similar descriptions apply, as will be apparent to a skilled person. The apparatus 120 may be used with the cover devices 50 described hereinbefore.

Referring now to FIGS. 9 to 12, the apparatus 120 comprises a collar portion 180 that fits around the magazine 122, preferably around the exterior of the magazine 122. In the preferred embodiment, the collar portion 180 extends around the entire periphery of the magazine 122 although, in alternative embodiments, not illustrated, one or more separate collar portions may be provided which extend only partially around the magazine 122. The collar portion 180 is advantageously located adjacent the mouth 124 of the magazine 122.

The collar portion 180 carries one or more projections 132 for dislodging the ring 56 of a cover device 50 from its seat 60. In the preferred embodiment where the collar 180 is located externally of the magazine 122, one or more apertures 182 are formed in the wall 184 of the magazine 122 so that at least the end portion 134 of the, or each, projection 132 may be located internally of the magazine 122, as may best be seen from FIGS. 10 to 12.

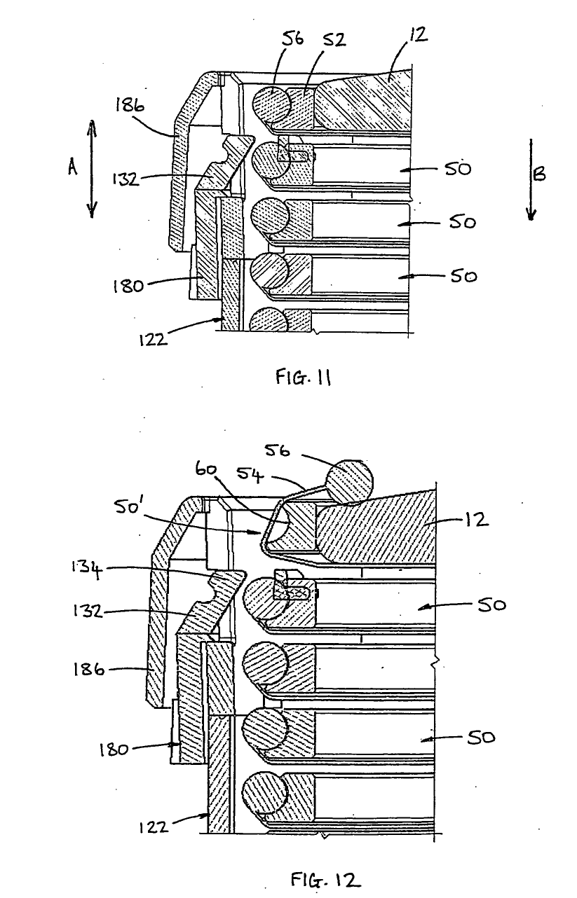

The collar 180 and the magazine 122 are moveable with respect to one another in a direction generally parallel with the longitudinal axis of the magazine, as illustrated by arrow A in FIG. 11. To this end, the collar 180 and magazine 122 may be coupled to one another in any convenient manner. Resilient biasing means, for example in the form of a spring (not shown), are provided between the collar 180 and the magazine 122 and arranged to urge the collar 180 and magazine 122 to move in said longitudinal direction and to adopt a rest state with respect to one another (as shown in FIGS. 9 to 12). In the rest state, the relative position of the collar 180 and the magazine 122 is such as to provide adequate space to receive the topmost cover device 50′ between the tip 134 of the projections 132 and the mouth 124 of the magazine 122 (see, for example, FIG. 11). In order to house the spring, or other biasing means, the magazine 122 may be provided with a flange 186 which at least partially surrounds the magazine 122 and is spaced-apart therefrom to receive at least part of the collar 180. Conveniently, therefore, the resilient biasing means may act on the flange 186 and on the collar 180.

During use, the apparatus 120 is installed in a support or carrier device (not shown) that may take the form of, for example, a ring or a sheath, such that the collar 180 is seated on the support device. A user (not shown) places a stethoscope head (or other article) against the topmost cover device 50′ as previously described. As the user pushes against the cover device 50′, the magazine 122, including the cover devices 50 contained therein, are moved in said longitudinal direction such that the mouth 124 and topmost cover device 50′ move towards the projections 132 (as indicated by arrow B in FIG. 11) against the bias of the resilient biasing means that is provided between the collar 180 and the magazine 122. As a result, the topmost cover device 50′ is moved towards the projections 132. Hence, the peripheral ring 56 of the cover device 50′ engages with the projections 132 and is dislodged from its seat 60 in the manner described above (see FIG. 12). Once the fitted cover device 50′ is removed from the apparatus 120, the resilient biasing means between the collar 180 and the magazine 122 urge the collar 180 and the magazine back to the rest state and the resilient biasing means (not shown) that actuates the platform 128 pushes the next cover device 50 past the projections 132 and into the topmost position.

The apparatus 120 may be easier to use than the apparatus 20 in cases where the magazine 122 holds a relatively large number of cover devices 50 since, in order to dispense the topmost cover device 50′, the user only has to overcome the bias of the resilient biasing means between the collar 180 and the magazine 122.

It will be appreciated from the foregoing that the invention provides means for quickly and easily dispensing covers for stethoscopes or other medical or non-medical instruments.

The embodiments described above relate to the diaphragm transponder 12 of a conventional stethoscope. In an alternative embodiment, not illustrated, the cover device and dispenser are arranged to be compatible with the bell transponder 16. In such an embodiment, it is advantageous for the membrane to comprise an aperture which, when fitted, is substantially in register with the open mouth, or aperture 17, of the bell transponder 16. Moreover, it will be understood that the invention is not limited to use with stethoscopes. Alternative embodiments of the invention may relate to the covering of the whole or part of other medical instruments or non-medical instruments.

The invention is not limited to the embodiments described herein which may be modified or varied without departing from the scope of the invention.

1. An apparatus for dispensing a cover device, the cover device comprising a frame and a membrane, the cover device being actuatable from a first state, in which an outer portion of the membrane is seated on an outer surface of the frame such that the frame holds the membrane in a generally deployed state, and a second state in which the outer portion of the membrane is located generally inwardly of its seated position, the apparatus comprising: a magazine for storing at least one cover device in said first state, the magazine having a mouth through which the at least one cover device may be dispensed; and means for actuating the at least one stored cover device from the first state to the second state.

2. An apparatus as claimed in claim 1, wherein said actuating means actuates the at least one stored cover device from the first state to the second state upon movement of said at least one stored cover device into the magazine.

3. An apparatus as claimed in claim 1, further including an actuating mechanism arranged to urge the at least one cover device towards the mouth.

4. An apparatus as claimed in claim 3, wherein said actuating mechanism comprises a platform mounted on a spring, the spring being biased to urge the platform towards the mouth.

5. An apparatus as claimed in claim 1, wherein a retaining lip is provided around at least part of the mouth, the retaining lip extending inwardly of the mouth.

6. An apparatus as claimed in claim 5, wherein the retaining lip is dimensioned to engage with, during use, the outer portion of the cover device nearest the mouth when in said first state, thereby retaining said cover device in the apparatus, and to allow said cover device to be removed from the apparatus when in the second state.

7. An apparatus as claimed in claim 1, wherein said actuating means comprises means for dislodging the outer portion of the membrane from its seat on the frame.

8. An apparatus as claimed in claim 7, wherein said dislodging means is actuatable between a dislodging state and a deflected state.

9. An apparatus as claimed in claim 8, wherein said dislodging means is actuatable from said dislodging state to said deflected state by passage of a cover device moving towards said mouth.

10. An apparatus as claimed in claim 8, wherein said dislodging means is biased to adopt the dislodging state.

11. An apparatus as claimed in claim 7, wherein said dislodging means comprises at least one projection extending away from an interior surface of the magazine and being spaced-apart from the mouth.

12. An apparatus as claimed in claim 11, wherein the spacing between the at least one projection and the mouth is such as to accommodate, during use, a single cover device between the at least one projection and the mouth.

13. An apparatus as claimed in claim 11, wherein said at least one projection extends obliquely from the interior surface of the magazine in a direction generally towards the mouth.

14. An apparatus as claimed in claim 11, wherein at least a part of said projections are formed from a flexible, resilient material.

15. An apparatus as claimed in claim 11, wherein said at least one projection is carried by a ring, the ring being seated on a shoulder formed at the interior surface of the magazine.

16. An apparatus as claimed in claim 7, wherein said dislodging means is spaced-apart from the mouth, the spacing being such as to accommodate, during use, a single cover device between the dislodging means and the mouth.

17. An apparatus as claimed in claim 1, the apparatus containing at least one of said cover devices.

18. An apparatus as claimed in claim 1, further comprising a collar, the magazine being movable with respect to the collar in a first direction, and wherein the collar carries said actuating means, said actuating means actuating the at least one stored cover device from the first state to the second state upon movement of said magazine in said first direction.

19. An apparatus as claimed in claim 18, wherein resilient biasing means are provided between said collar and said magazine, the resilient biasing means being arranged to urge said magazine in a second direction, said second direction being generally opposite to said first direction.

20. An apparatus as claimed in claim 18, wherein said collar is located at least partially around the external periphery of the magazine.

21. A cover device comprising a frame and a membrane, the cover device being actuatable from a first state, in which an outer portion of the membrane is seated on an outer surface of the frame such that the frame holds the membrane in a generally deployed state, and a second state in which the outer portion of the membrane is located generally inwardly of its seated position.

22. A cover device as claimed in claim 21, wherein the outer portion of the membrane comprises a peripheral ring.

23. A cover device as claimed in claim 21, wherein the outer surface of the frame is concave in transverse cross-sectional profile to define a seat for the outer portion of the membrane.

24. A cover device as claimed in claim 21, wherein at least the outer portion of the membrane is formed from elastic material.

25. A cover device as claimed in claim 24, wherein, in the first state, said outer portion is stretched by the frame such that the membrane is self-retaining on the frame.

26. A cover device as claimed in claim 25, wherein, in the second state, said outer portion is contracted with respect to the first state and is located generally inwardly of the frame.

27. A cover device as claimed in claim 21, wherein the outer surface of the frame is shaped to define a seat for the outer portion of the membrane.
